(19) United States
(12) Patent Application Publication
(10) Pub. No.: US 2009/0298097 A1
(43) Pub. Date: Dec. 3, 2009

(54) METHODS FOR THE DIAGNOSIS OF LUNG CANCER

(76) Inventors: Pamela J. Harris, Bethesda, MD (US); Michael S. Lebowitz, Baltimore, MD (US); Hossein A. Ghanbari, Potomac, MD (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/175,451

(22) Filed: Jul. 17, 2008

Related U.S. Application Data
(60) Provisional application No. 60/959,869, filed on Jul. 17, 2007.

(51) Int. Cl. G01N 33/574

(52) U.S. Cl. 435/7.23

(57) ABSTRACT

The present invention is directed to new ways to diagnosis lung cancer, especially at an early clinical stage. In addition, prognosis and the monitoring of therapeutic agents or other treatments, for lung cancer patients, can be accomplished with the disclosed methods. The methods also find use in allowing the assessment by pre-clinical animal efficacy studies to screen for the useful of therapeutic agents for treating lung cancer.

This application claims priority from U.S. provisional application No. 60/959, 869, filed Jul. 17, 2007.

### FIELD OF THE INVENTION

The present invention is related to lung cancer diagnosis, as well as the collateral aspects of the prognosis and the monitoring of therapeutic agents or other treatments, for lung cancer patients. The methods are also useful in pre-clinical animal efficacy studies to screen for the useful of therapeutic agents for treating lung cancer.

### BACKGROUND OF THE INVENTION

It is projected that more than 170,000 cases of lung cancer will be diagnosed in the United States in 2007, and such detection will occur by means such as X-ray and CT scanning methods, which have inherently low sensitivity (for early cancer stages especially) and high cost, as compared to serological types of diagnostic methods, generally.

While the five year survival for lung cancer is generally estimated at 15%, a survival rate of about 50% can be achieved when detection is made early in individuals with localized cancer. The current detection methods, however, enable such detection in only about 16% of cases overall.

Lung cancer is a leading cause of cancer, and associated with high mortality worldwide. To date, there are no known blood/serum biomarkers useful for the diagnosis of lung cancer, or at least that are currently approved by regulatory authorities for the detection of such cancer. Current detection methods are, comparatively, inadequate to address the public health issues concerning lung cancer.

Despite advances made in the diagnosis and treatment of a number of common cancers, lung cancer remains a leading cause of cancer death worldwide. A primary factor contributing to the high mortality rates associated with lung cancer is the lack of early diagnosis, prior to significant advancement and associated symptoms of the disease. Thus, the majority of lung cancer patients are not identified until they have developed stage III or IV tumors, which are largely not well treated by surgical resection or by radiation, chemo- and biological therapies. Thus, early determination of the presence of lung cancer would greatly enhance therapeutic success and lessen mortality rates from this devastating disease.

Surgery for patients with Stage I and IIa lung cancer can be curative and it is critical to identify these patients before their cancers have progressed. Unfortunately, current screening tests to identify lung/bronchial cancer in its early stages have been disappointing and none of the nationally recognized medical or oncological associations including the American College of Chest Physicians (ACCP), the National Comprehensive Cancer Network (NCCN), the American Society of Clinical Oncology (ASCO) and the American Cancer Society (ACS) have recommended a specific screening regimen for the early detection of lung cancer even for individuals at high risk. While a minority of specialists recommends annual low-dose spiral computed tomography (CT) scans for screening of people at high risk, the utility of such screening has yet to be accepted despite the recent results of a very large clinical trial by the International Early Lung Cancer Action Program (I-ELCAP). In general, while CT scanning can identify some cases of lung cancer early and at a curable stage, its application is impractical, expensive and has been associated with false positive results leading to inappropriate surgical interventions.

Similarly, there is no biomarker for surveillance of lung cancer patients treated with curative intent. The current recommendations of the NCCN for surveillance of these patients for lung cancer recurrence include history and physical and contrast enhanced CT scanning of the chest every 4-6 months for the first 2 years and then history and physical with non-contrast enhance CT of the chest annually thereafter (Ettinger D S et al., “Non-small cell lung cancer clinical practice guidelines in oncology.” J Natl Compr Canc Netw 4:548-82, 2006). Blood tests, PET scanning, sputum cytology, tumor markers and fluorescence bronchoscopy are specifically not recommended (Alberts W M: Diagnosis and management of lung cancer executive summary: ACCP evidence-based clinical practice guidelines (2nd Edition). Chest 132:1S-19S, 2007).

A potential key to improving the early detection of lung cancer is the identification of an inexpensive, minimally invasive test that could further identify those individuals who might benefit from further diagnostic follow-up. Such a test might be useful either as a guide to the interpretation of CT scan results or as an initial screen suggesting further work-up which might include CT scanning. Application of such a test to the specific case of lung cancer recurrence adds further benefits in that it may identify metastatic disease that has recurred outside of the lung and thus may be missed by chest CT. While a number of tumor markers have been explored, including cytokeratin fragments (CYFRA 21-1), neuron specific enolase (NSE), prograstin releasing peptide (ProGRP), squamous cell carcinoma antigen (SCC), carcinoembryonic antigen (CEA) and tumor M2-pyrvate kinase (Tumor M2-PK), to date none of these have demonstrated the requisite sensitivity and/or specificity to be clinically meaningful (Greenberg A K, Lee M S: Biomarkers for lung cancer: clinical uses. Curr Opin Pulm Med 13:249-55, 2007; and Schneider J: Tumor markers in detection of lung cancer. Adv Clin Chem 42:1-41, 2006). While some researchers have begun to apply serum biomarker panels for the diagnosis of lung cancer, the best of these have demonstrated sensitivities of 78% and specificities of only 75% (Patz E F, et al: Panel of serum biomarkers for the diagnosis of lung cancer. J Clin Oncol 25:5578-83, 2007).

Thus, a major challenge in the field of lung cancer therapy lies in the accurate diagnosis of the disease at a stage early enough to allow, or optimize, successful treatment. Moreover, a simple, non-invasive test that would allow clinicians to monitor the disease during treatment with one or more therapeutic modalities/agents would bode well for the patient's recovery. Still further, a way to test the effectiveness of therapeutics pre-clinically would allow a swifter avenue to the market for promising drugs. There clearly exists a need for improved methods and reagents for accomplishing these goals.

### SUMMARY OF THE INVENTION

The enzyme, aspartyl (asparaginyl) β-hydroxylase (“AAH”), has been shown to be overexpressed in many malignant tumors of endodermal origin and in at least 95% of CNS tumors compared to normal noncancerous cells. Previous work has shown that AAH is overexpressed on the surfaces of lung cancer cells, for instance. See further, Wands et al., U.S. Pat. Nos. 6,797,696; 6,783,758; 6,812,206; 6,815,415; 6,835,370; and 7,094,556, each of which is hereby incorporated by reference in its entirety.

It has now been found that human AAH (also referred to as “HAAH” or “ASPH” herein and in the prior art) is not only overexpressed in lung cancer cells per se, but is present in the cancerous subject to such an extent that a diagnosis of a lung cancer condition is discernable by assaying for, or detecting, HAAH in the blood (which includes, in accordance with this disclosure, blood components, e.g. serum and plasma, but which is preferably serum) of a human subject. While prior publications have established that AAH is an exceptional cell surface biomarker for malignancies, and that its diagnostic value is also well-associated with bodily fluid levels, the present invention reveals for the first time a clear correlation between blood levels of AAH and lung cancer, and that these levels can be determinative as a screening tool, as a diagnostic tool adjunctive to state of the art tests, and even as a quick and non-invasive test to monitor therapy.

This discovery has many implications. In accordance with the present invention, AAH is an excellent biomarker for lung cancer detection, especially at an early stage in which the cancer is most responsive to therapy, as well as a tool for drug discovery, and as a marker for monitoring efficacy of treatment (drug or other) in a lung cancer patient.

The cancer biomarker, aspartyl (asparaginyl) β-hydroxylase, has previously been found to be elevated by in a broad range of cancers, including lung cancer, by immunohistochemical staining (IHC) of cancerous tissues. Human AAH (or HAAH) was detected in >99% of tumor tissue specimens tested (n>1000), yet absent in adjacent, normal, tissue.

The present invention provides evidence for a correlation, or link, between levels of human AAH detected in blood (including serum, for instance) and the presence (or not) of lung cancer in a human patient.

Thus, the present invention provides for methods for identifying whether a subject has lung cancer, comprising contacting a serological sample of the subject with an anti-AAH antibody in vitro, under conditions that will allow immunological binding to occur, and detecting the level of anti-AAH/AAH immunocomplexes thus formed, whereby a detectable level of AAH in the serological sample is indicative of the presence of lung cancer in the subject.

The HAAH serum immunoassay therefore has great promise as an additional diagnostic tool for lung cancer having the practicality and cost effectiveness of conventional serological screening. Elevated serum HAAH in conjunction with CT scanning, the current state of the art in diagnostics for lung cancer, may greatly facilitate earlier diagnosis of lung cancer at a stage in which cure rates are significantly higher and thus may contribute to increased patient survival.

In view of the present discovery of a determinative marker for lung cancer in a serological sample, and that this marker can be a powerful diagnostic tool at the early stages of lung cancer, clinical evaluations can be more accurately assessed and treatments performed at a point in time that should allow a higher success of intervention and treatment. Concurrently, the detection of AAH, quantitatively or in a positive/negative manner, allows the clinician a tool to assess responses to various therapeutic regimens/agents, and can be used as a guide for prognoses in lung cancer patients. For instance, used as a prognostic tool, the assay of AAH will allow for rational choices of the best course and best drug(s) to be used in therapeutic interventions, and direct patients to the most appropriate treatments.

Moreover, AAH levels in serological samples and the like may be used to screen for potentially effective therapies/drugs against lung cancer. Thus, the present invention also provides a method for screening potential therapeutic agents by measuring the expression of AAH (or HAAH) in blood samples as compared to corresponding samples from normal, non-cancerous controls.

### BRIEF DESCRIPTION OF THE DRAWINGS

FIG. 1 is a graph depicting HAAH levels in serum from patients with confirmed diagnoses of NSCLC (n=160) and control individuals not known to have cancer (n=93), as described in Example 1.

FIG. 2 shows the comparison of HAAH levels in serum from patients with confirmed diagnoses of NSCLC (n=163) and control individuals not known to have cancer (n=43), as described in Example 2 (initial sample set). The mean values for the two groups were 33.8±2.1 ng/ml (range −1.5 to 148 ng/ml) and −0.7±0.4 ng/ml (range −3.5 to 8.5 ng/ml), respectively. The inset depicts a focus on the lower range of HAAH value (0 to 50 ng/ml) which clearly delineates the significant difference in the HAAH levels of the two groups of patients.

FIG. 3 depicts the sensitivity and specificity of the above assay as plotted over a range of potential cutoff values. The optimal cutoff was determined to be 3.2 ng/ml to maximize the sensitivity (93%) and specificity (95%) of the test.

FIG. 4 shows the HAAH levels in lung cancer patients separated by stage (n=15 for each stage) and compared to individuals at high risk for lung cancer (i.e. smokers; n=50), as described in Example 2 (secondary sample set). Mean values±the standard error of the mean are shown for each stage of lung cancer and for the group of smokers.

FIG. 5 depicts the sensitivity and specificity of the assay including all test samples (n=223 cancers and n=93 non-cancers) and plotted over a range of potential cutoff values. The optimal cutoff was determined to be 3.7 ng/ml to maximize the sensitivity (94%) and specificity (94%) of the assay.

### DETAILED DESCRIPTION OF THE INVENTION

We and others have shown in previous studies using immunohistochemical staining of paraffin-embedded sections of human lung cancer tissue specimens showed a distinct, elevated presence of HAAH in these tissues (and not in surrounding, normal tissue). It was unknown, however, if HAAH would be measurable at all in serological samples of patients with lung cancer.

The detectable presence of AAH in the blood (which includes components thereof, such as plasma and serum) is a valuable tool not only for diagnosis, but for assessing whether a given patient is responding to treatment with a particular drug or treatment, and for screening candidate drugs/therapies in a broader sense, because decreases in (human) AAH levels in the blood of a lung cancer patient can be correlated with drug/therapy success.

One aspect of the present invention relates to methods for detecting lung cancer in a serological sample, including as a confirmatory diagnosis of the disease, for disease progression, relapse, or remission. Such methods comprise determining if AAH is overexpressed (i.e., has a higher AAH protein level) in a serological test sample as compared to a normal sample. In a preferred embodiment, the sample is serum. In practical terms, the methods provide a way to determine the presence or absence of AAH, with the measurable presence of AAH being indicative of a lung cancer state More specifically, the present invention provides for such methods as: (1) identifying whether a subject has a lung cancer condition, by contacting a serological sample of the subject with an anti-AAH antibody in vitro, allowing a period of time for any specific immunological binding to occur with AAH in the sample, and detecting the level of anti-AAH/AAH immunocomplexes thus formed (with a detectable level of AAH being indicative of the presence of lung cancer in the subject; (2) allowing for the clinical monitoring of a therapeutic treatment of a lung cancer patient by assaying a first serological sample at a first time point from the patient prior to or at some point during treatment, and repeating the assay at predetermined or otherwise desired time intervals during (or after) treatment, whereby changes in expression of HAAH are correlated with clinical performance of the treatment (clearly with higher HAAH levels indicating failure, and vice versa for success).

As some preferred embodiments of the invention, the level of AAH is determined by an ELISA immunoassay format, and in any case is preferably an immunological assay that employs two homologous antibodies, one of which is biotinylated. Further, the invention finds particular utility in humans, and as such human samples and detection of human AAH are preferred embodiments.

In cases of screening for lung cancer or for confirming diagnosis as an adjunct test, a positive result is indicated by a serum level of AAH about 3.2 ng/ml or higher, and more preferably 3.7 ng/ml or higher. These cutoff points have been determined through the studies of the invention, and can in fact be employed to analyze positive/negative results in serum samples.

The present invention also provides a method to determine pre-clinically whether a potential therapeutic agent is an effective drug for the treatment of lung cancer, by establishing a human lung cancer tumor in a laboratory animal model in a known manner, obtaining a serological sample from the animal model at a time prior to instituting treatment with the test therapeutic agent, detecting the level of AAH in said sample by assaying for human AAH protein with an immunological test format using one or more anti-HAAH antibodies, and repeating this test one or more times over a course of treatment with the therapeutic agent, and observing whether HAAH levels are increased or decreased during the time course, with decreases indicating effectiveness and vice versa for increases Of course, the methods of determining HAAH levels in the serological samples are any of those mentioned herein.

The level of AAH in a patient-derived blood sample is carried out using any standard methodology that measures levels (as compared to known normal controls) of a certain protein, e.g., by Western blot assays or a quantitative assay such as ELISA. In this case, the protein to be determined is AAH. While ordinarily such quantitative tests are performed alongside normal controls, the inventors have determined by the studies described herein certain cutoff points (3.2 ng/ml and 3.7 ng/ml in two studies), above which a sample can be indicated as positive for the disease. Thus, for instance, in a simple AAH protein level determination in a serum sample, the sample need not be run concurrently with a normal control sample.

For an example of an assay format, a standard competitive ELISA using an (human) AAH-specific antibody is used to quantify human patient HAAH (i.e., human AAH) levels. Alternatively, a sandwich ELISA using a first antibody as the capture antibody and a second HAAH-specific antibody as a detection antibody can be used.

As some preferred embodiments, the level of AAH is preferably determined by an ELISA immunoassay format, and in any case employs two homologous antibodies, one of which is biotinylated. Further, the invention finds particular utility in humans, and as such human samples and detection of human AAH are preferred embodiments.

Methods of detecting AAH also include contacting a serological sample with an AAH-specific antibody bound to solid matrix, e.g., microtiter plate, bead, dipstick. For example, the solid matrix is dipped into a patient-derived blood sample (or component thereof), washed, and the solid matrix is contacted with a reagent to detect the presence of immune complexes present on the solid matrix.

The nature of the solid surface may vary depending upon the assay format. For assays carried out in microtiter wells, the solid surface is the wall of the well or cup. For assays using beads, the solid surface is the surface of the bead. In assays using a dipstick (i.e., a solid body made from a porous or fibrous material such as fabric or paper) the surface is the surface of the material from which the dipstick is made. Examples of useful solid supports include nitrocellulose (e.g., in membrane or microtiter well form), polyvinyl chloride (e.g., in sheets or microtiter wells), polystyrene latex (e.g., in beads or microtiter plates), polyvinylidine fluoride (known as IMMULON®), diazotized paper, nylon membranes, activated beads, and Protein A beads. The solid support containing the anti-AAH antibody is typically washed after contacting it with the test sample, and prior to detection of bound immune complexes. Incubation of the antibody with the test sample is followed by detection of immune complexes by a detectable label. For example, the label is enzymatic, fluorescent, chemiluminescent, radioactive, or a dye. Assays which amplify the signals from the immune complex are also known in the art, e.g., assays which utilize biotin and avidin.

Anti-AAH antibodies useful for AAH detection are, for example, those disclosed in the patents of Wands et al., supra (which are produced by hybridomas that have been deposited with the American Type Culture Collection as accession numbers PTA 3383, PTA 3384, PTA 3385, and PTA 3386), including fragments and derivatives (e.g., labeled) thereof. As a preferred embodiment, and used in the Examples, is the antibody referred to herein as FB50, which is produced by the hybridoma PTA 3386.

In another aspect of the invention, an AAH-detection reagent for the detection of AAH for screen or diagnosis of lung cancer, e.g., one or more anti-AAH antibodies (or immunologically reactive fragments or derivatives thereof), may be commercially distributed alone, or packaged in the form of a kit with other items, such as control formulations (positive and/or negative), and a detectable label. As a correlative aspect is a device containing such reagent deposited on a solid surface, such as a dipstick or biochip or the like. The assay to be used with such test kit may be in the form of any standard homologous or two-antibody sandwich assay format known in the art. But, minimally, a test kit or device for the immunological determination of a level of AAH protein in a serological sample from a patient known or unknown to have lung cancer has at least one anti-AAH antibody that which will specifically bind to AAH, and preferably, instructions for performing the test and interpreting the results.

Concerning a device, such as a dipstick or biochip, it is contemplated that such a device would be comprised of a solid support, onto which the one or more specifically binding anti-HAAH antibodies is (are) covalently bound, and wherein at least one of these bound antibodies is labeled with a detectable label. Further situated within said dipstick or biochip device is one or more compounds with which to detect the detectable label of the antibody after its determined time of contact with a serological sample from a patient suspected of or known to have lung cancer. Thereby, the formation of immunological complexes of the anti-HAAH antibody and HAAH in the serological sample, visually or otherwise, can be detected and a positive or negative result determined accordingly. A device of the invention preferably has one anti-HAAH antibody, which is detectably labeled, and which acts as both capture and detection antibody.

While methods for directly diagnosing lung cancer are well practiced in the art, the diagnostic method of the present invention can be an adjunct to initial diagnosis, or may be used as a screening tool for to identify patients within the early clinical stages of the disease. The methods may also be used to monitor recurrence or remission of the cancer in treated patients at regular intervals or at the desire of the patient or physician.

In another aspect of the present invention, one can use an assay for AAH in the blood in accordance with the present invention to determine if an individual patient responds to a particular drug or treatment regimen. In this aspect of the present invention, there is provided a method for monitoring a course of a therapeutic treatment in an individual being treated for lung cancer, comprising: a) obtaining a blood sample at a first time point from a patient having said treatment; b) measuring the levels of AAH by assaying for AAH protein in the sample; and c) repeating steps a) and b) at determined time points during the course of treatment, whereby the therapeutic treatment is temporally monitored by detecting any changes in the levels of AAH, and wherein a decrease AAH over time is associated with the success of the therapeutic treatment, and increased AAH levels are indicative of failure of the treatment. Of course, ever-increasing AAH levels over time in the course of treatment may be indicative of acquiring non-responsiveness to a drug and reason to change therapeutic modalities.

The assays of the present invention may also be used in pre-clinical animal studies of therapeutic agents for lung cancer to screen for effectiveness of the drugs in a manner such as described in the paragraph above.

A therapeutic agent to be evaluated for effectiveness, either pre-clinically or during treatment of lung cancer, by an assay of AAH is not limited to any particular substance or class, and may be, for instance, a small molecule, an antibody, or an antisense polynucleotide.

The assay format described below may be used to screen potential anti-lung cancer agents or to generate temporal data used for long-term therapeutic effectiveness or prognosis of the disease. For example, an assay for AAH protein is carried out, in general, by contacting a blood (or serum or plasma) sample from a mammal (as a preferred embodiment, a human) with an antibody that specifically binds to an AAH polypeptide under conditions sufficient to form an antigen-antibody complex, detecting the antigen-antibody complex, and quantitating the amount of complex to determine the level of AAH in the sample.

Anti-AAH antibodies useful for AAH detection are, for example, those disclosed in the patents of Wands et al., supra, which are produced by hybridomas that have been deposited with the ATCC under accession numbers PTA 3383, PTA 3384, PTA 3385 and PTA 3386, including fragments and derivatives (e.g., labeled) thereof.

An AAH-detection reagent, e.g., one or more anti-AAH antibodies (or immunologically reactive fragments or derivatives thereof), may be commercially distributed alone, or packaged in the form of a kit with other items, such as control formulations (positive and/or negative), and a detectable label for use in the methods of the present invention. Such an assay to be performed with such kits may be, e.g., a standard two-antibody sandwich assay format known in the art.

An assay for AAH in a (suspected or confirmed) lung cancer subject can be used to diagnose the disease, measure efficacy of a drug candidate, or chart the prognosis or the effectiveness of a course of therapy over time. An increasing level of AAH over time indicates a progressive worsening of the disease, and therefore, an adverse prognosis, or lack of (or continuing) effectiveness of the therapy.

The assay used in the examples below is of an ELISA format, which is a format well known to those in the art. The sequences of the HAAH polypeptide and the HAAH cDNA are known from, inter alia, U.S. Pat. No. 6,835,370 and related patents thereof, as well as other prior art, and the knowledge conveyed by these disclosures will allow one of ordinary skill to readily determine and obtain the assay reagents for this and other assay types. Thus, the present invention is not limited to any particular assay reagents or format, as long as AAH protein expression is the measurable endpoint to surveil lung cancer.

While methods for directly diagnosing lung cancer are available and currently practiced in the art, the diagnostic method of the present invention can be an adjunct to initial diagnosis, or may be used quantitatively to assess a clinical stage of the disease. As such, as the illness progresses, increasing amounts of AAH expression (i.e., protein levels) in periodic serological test samples of a patient over time would be indicative of a worsening disease state; conversely, decreasing amounts of AAH expression would indicate improvement of the patient's condition.

In another aspect, the present invention provides a method for screening potential therapeutic agent(s) or other therapeutic modalities (such as radiation) for lung cancer. Essentially, the method comprises collecting serological samples from a lung cancer patient receiving such treatment/drug(s), and measuring AAH protein levels in such sample (or samples taken over a course of time), and comparing such level(s) to a corresponding protein level of AAH of a control sample.

The therapeutic agent being evaluated is not limited to any particular substance or class, and may be, for instance, a small molecule, a peptide, an antibody, or an antisense polynucleotide. Interestingly, the candidate being evaluated does not necessarily have to interact with AAH directly; the successful candidate need only have an indirect negative modulation (i.e., inhibitory effect) on AAH expression or activity. The amount of AAH protein in the samples can also be measured by any available method that measures levels of a specific protein in a sample, such as immunological assays or protein separation techniques. The basic principle of this aspect of the invention is to identify compounds that inhibit AAH protein levels in the blood of test samples.

In yet another aspect of the present invention, there is provided a method for monitoring a course of a therapeutic treatment in an individual being treated for lung cancer, comprising: a) obtaining a blood sample at a first time point from a patient undergoing said treatment; b) detecting or quantitatively assaying for (human) AAH protein; and c) repeating steps a) and b) at determined time points during the course of treatment, whereby the therapeutic treatment is temporally monitored by detecting any changes in expression of the HAAH gene, and wherein the decreased expression of the HAAH gene is associated with the success of the therapeutic treatment, and increased HAAH gene expression is indicative failure of the treatment. Of course, ever increasing HAAH expression over time in the course of treatment is indicative of acquiring non-responsiveness and reason to change therapeutic modalities.

As mentioned previously, one may use immunological methods with labeled antibodies to AAH to detect levels of AAH protein. The methods for analyzing or measuring AAH are conventional and well known to those skilled in the art or may be readily implemented without undue experimentation.

With all the methods of the present invention, in monitoring treatment or assessing relapse, it is understood that the monitoring should be done in a consistent manner and that the treatment being assessed is not an anti-AAH antibody treatment, if the assay used is of an immunological format for the polypeptide.

The assay format described in the Examples below may be used to diagnose lung cancer or to generate temporal data used for long-term therapeutic effectiveness or prognosis of the disease.

The invention is further illustrated by the following examples, which are not intended to limit the scope of the appended claims.

### EXAMPLES

### Example I

A large study was conducted using a double monoclonal (FB50 anti-HAAH antibodies) sandwich-type ELISA format with anti-AAH antibodies, providing detection and comparative quantification of HAAH in serum samples obtained from lung cancer patients, and control samples obtained from a pool of non-cancerous subjects (which included samples of cigarette smokers as well—“high risk controls”). The high risk controls were relevant to this study, because 87% of lung cancers are attributable to cigarette smoking, and associative parallels with recent reductions in rates of smoking have been reported in the literature.

### Results

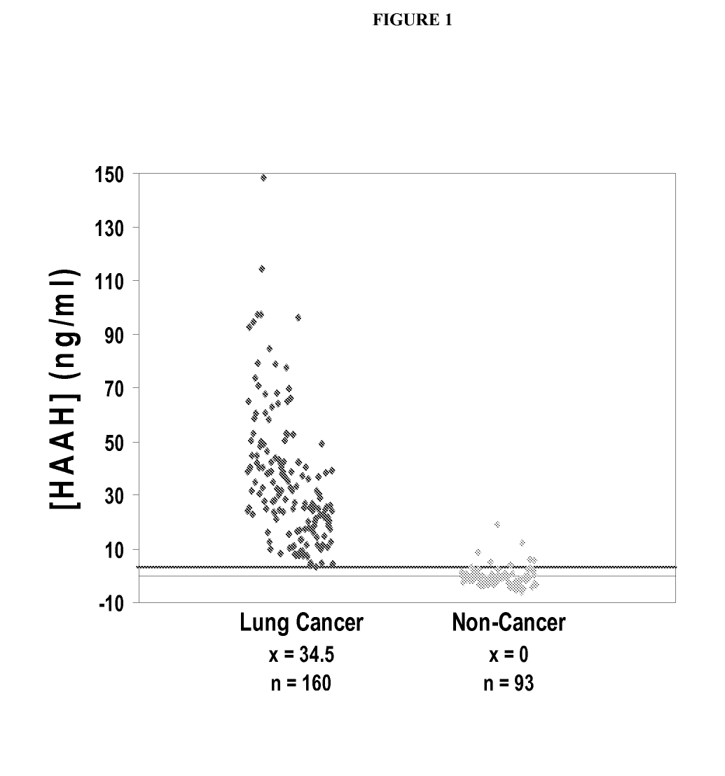

Increased levels of serum HAAH were found in 99% of patients with lung cancer (n=160). Quite strikingly, serum HAAH levels were found to be undetectable in individuals not known to have cancer (normal controls) (n=93, specificity=91%). See FIG. 1.

In the control subpopulation of 50 smokers not known to have cancer, the mean serum HAAH level was 0 ng/ml, with 90% specificity.

The results of the serum ELISA for AAH show that this assay for AAH is very predictive of a lung cancer state in an unknown sample. Coupled with CT scanning, which is the state of the art, an earlier diagnosis of lung cancer at a stage in which cure rates are significantly higher, which is achievable using this assay for AAH levels in the blood (serum/plasma), will contribute to an increased patient survival rate.

### Example 2

The goal of this study was to provide further evidence of the clinical sensitivity and specificity of the diagnostic test of the present invention, as well as its unique utility as a screen for NSCLC (non-small cell lung cancer) in patients at increased risk of this disease, due to its ability to detect lung cancer as early as stage I of the disease.

Briefly, sera obtained from patients with a confirmed diagnosis of NSCLC (n=163) and individuals with no known history of cancer (n=43) were analyzed in a homologous antibody immunoassay for the detection of HAAH and used to determine a threshold value for the test that could serve to discriminate between subjects with and without NSCLC. A second set of patients, including subjects with stages I-IV NSCLC (n=60) and a control group of subjects with a history of moderate to heavy smoking (n=50), was used to further establish this cutoff value.

Briefly, the resulting data showed that sera from individuals with NSCLC had elevated levels of HAAH ( x̅=29.9 ng/ml) as compared to those from individuals not known to have cancer ( x̅=<2 ng/ml). A cutoff value was therefore established to be 3.7 ng/ml, which provided a sensitivity of 94% and a specificity of 94% for the test. Particulars of this study are described below.

Anti-HAAH antibody (FB50), biotinylated FB50, and recombinant HAAH were all prepared in manners previously reported (in the patents, supra, among other prior publications) and for this study were produced by our laboratory.

163 sera from individuals with a confirmed diagnosis of NSCLC were obtained from several repositories in the US. One hundred of these samples included 51 females and 49 males with an average age of 77 years; no other information for the remaining 63 samples was known, except for a positive diagnosis of lung cancer. The control set comprised 43 samples of sera from males over the age of 50 and not known or suspected to have cancer.

ELISA to detect the presence of HAAH in human serum. The HAAH ELISA was carried out with the monoclonal antibody FB50 in a homologous format using the same antibody for both capture and detection steps. The FB50 antibody is produced by the hybridoma cell line on deposit with the ATCC, designated accession number PTA 3386, and has been described previously in the Wands et al. patents, supra. Recombinant HAAH was prepared as an affinity purified baculovirus-expressed protein and served as an assay calibrator.

Serum samples, standards, and controls were first diluted 1/10 v/v with assay buffer and heated at 50° C. for 30 minutes in sealed polypropylene 96 well blocks (NUNC). Flat bottom high binding 96 well polystyrene microplates (Costar) were coated with the FB50 monoclonal anti-HAAH antibody in 0.2 M sodium bicarbonate coating buffer at 2 μg/ml. A one hour 37° C. coating step was followed by aspiration, two washes, and blocking with 1% BSA blocking buffer for 1 hour at 37° C.

The treated serum samples were transferred to the coated/blocked microtiter plates and incubated at 37° C. for 2 hours. In a sequential fashion, with intervening wash steps, the plates were then incubated with biotinylated FB50 antibody for 1.5 hours at 27° C., followed by incubation with peroxidase-streptavidin ( 1/5000 v/v) for 45 minutes, and finally incubating with TMB substrate. Reactions were terminated with 2.5N sulfuric acid, and the plates were read at 450 nm. Results were interpolated with the standard curve to calculate values of unknown samples. All determinations were performed in triplicate.

### Results

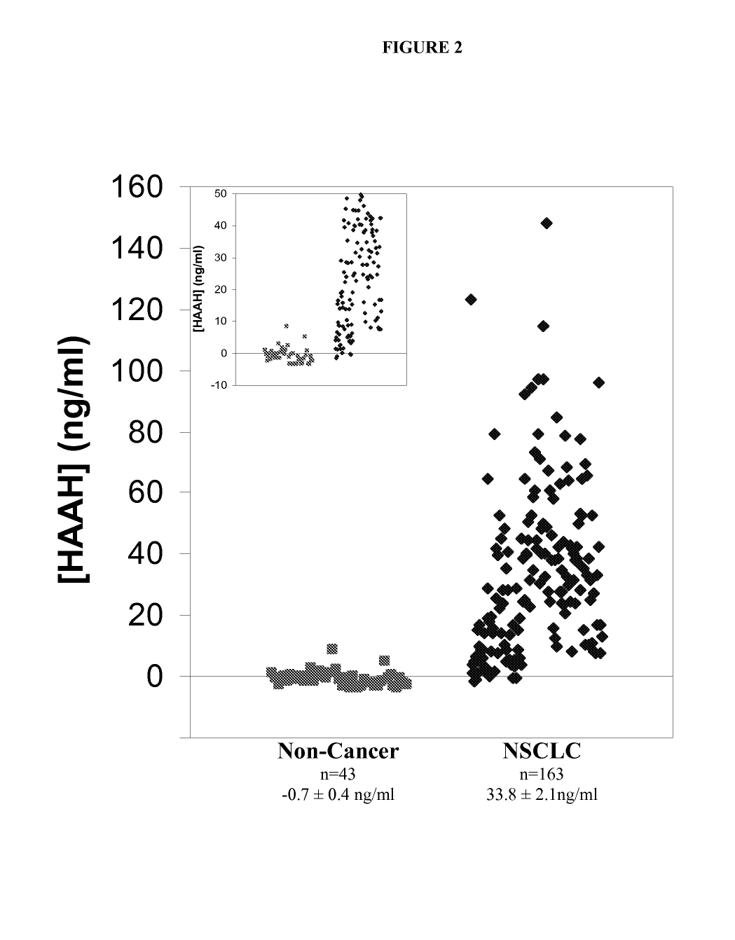

Initial serum test panel. All sera samples (test and control) were analyzed with the HAAH ELISA and results are shown in FIG. 2. The vast majority, 154 out 163 (94%), of test samples had significantly detectable HAAH levels (≧2 ng/ml) compared to only three of the 43 (7%) control serum samples.

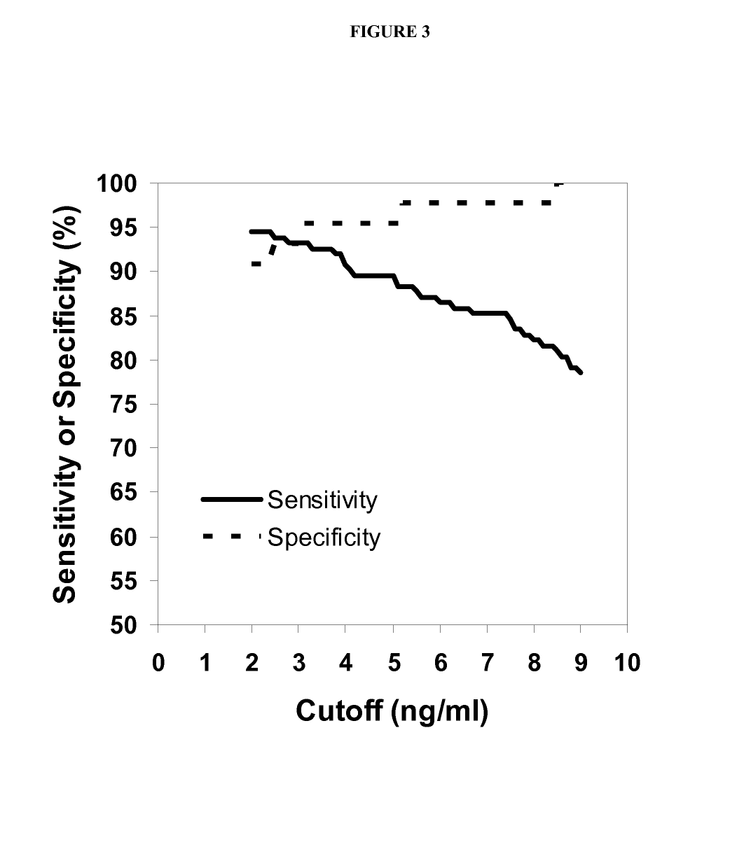

The mean HAAH level for the test sera was 33.8±2.1 ng/ml (with a range −1.5 to 148 ng/ml) and for the non-cancer sera was −0.7±0.4 ng/ml (range of −3.5 to 8.5 ng/ml). It is clearly evident from the results that HAAH is significantly elevated in the serum of individuals with NSCLC as compared to controls. A plot of the sensitivity and specificity of the assay as a function of a threshold value in ng/ml indicates an optimal cutoff set at 3.2 ng/ml (sensitivity=93% and specificity=95%) (FIG. 3). In other words, the set cutoff value of the test is at or near the level of detection of AAH in this assay, and this means that this test can be used to depict a positive or negative result without having to specifically measure the concentration of HAAH in the sample.

Validation of the HAAH assay (secondary data set). In order to validate the HAAH assay as a clinically significant diagnostic tool for non-small cell lung cancer, we further obtained a set of 60 samples from individuals with a confirmed diagnosis of lung cancer for whom staging information was known. The test serum samples were obtained from patients with known NSCLC and known staging of the illness (n=15 for each stage I, II, III and IV). The control group was of 50 serum samples collected prospectively from “heavy” smokers, who were recorded as never having been diagnosed with cancer. Information regarding the smoking habits of these control group individuals was also recorded.

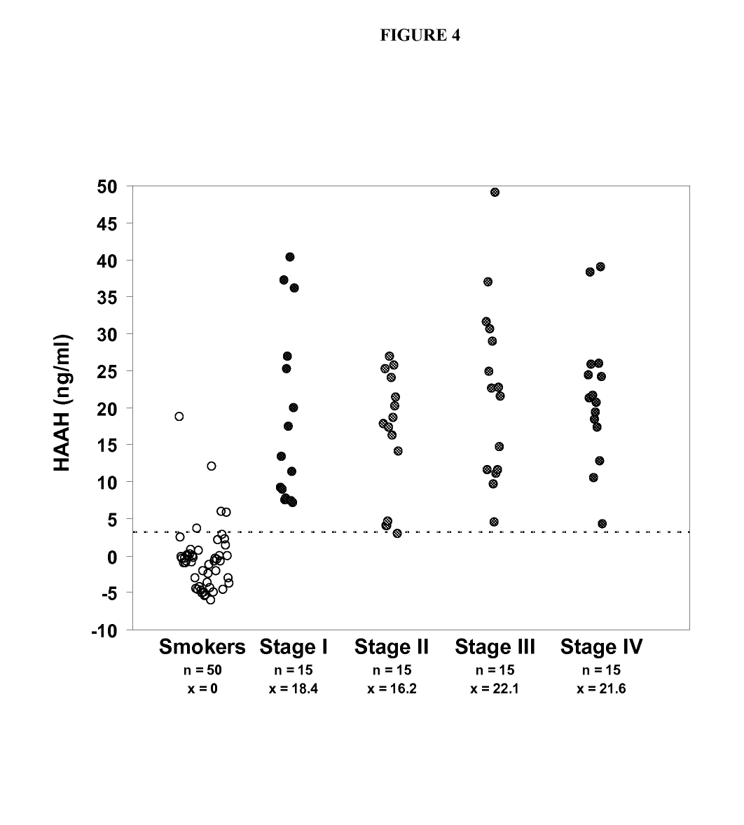

As can be seen in FIG. 4, HAAH values were elevated in all stages of NSCLC, including stages I & II. These test group HAAH levels did not correlate with cancer stage, but rather with the presence or absence of disease. The average HAAH value in the serum of the control smoker population was −0.6±0.6 ng/ml compared to an average value in the cancer samples (all stages) of 19.6±1.4 ng/ml.

Application of the 3.2 ng/ml cutoff identified from the initial serum panel to the validation set yielded a sensitivity of 98% and a specificity of 90% for the assay. This mild decrease in specificity may be related to the smoking status of the control group in the second data set, but may also be an indication of an as yet undiagnosed cancer in several individuals within the control group of smokers.

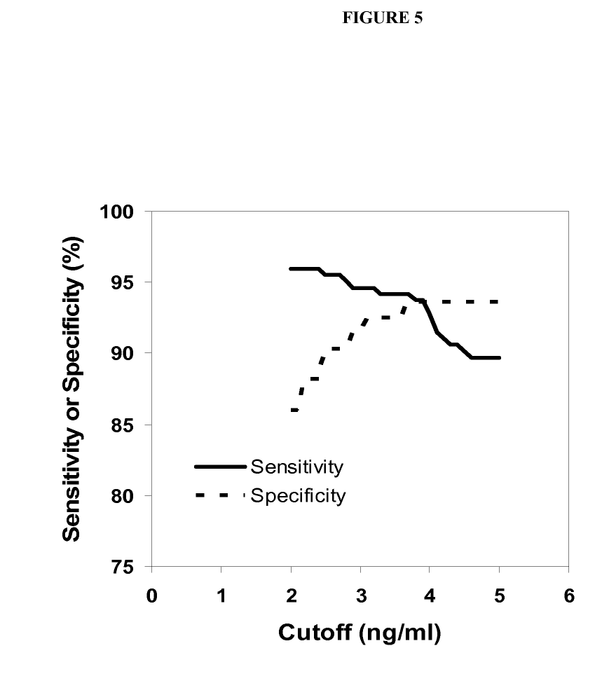

Taking both initial and secondary sample sets together, and plotting the sensitivity and specificity of the assay as a function a threshold value in ng/ml, indicates an optimal cutoff of 3.7 ng/ml, which correlates to an overall sensitivity and specificity of 94% for each of these statistical determinations (FIG. 5).

The sensitivity of the HAAH serum test for NSCLC was essentially the same in all clinical stages of the disease, and in fact the absolute levels of serum HAAH (both mean and median) were the same for all stages. It can therefore be inferred that the diagnostic levels of HAAH in the serum of lung cancer patients is both an early event in disease development as well as a persistent feature of the malignancy. While these experiments have not established a firm basis for clinically staging lung cancer, they indeed establish a way to diagnose lung cancer in a serological sample at an early stage (stage I or II), at which point a patient may not have any other apparent symptoms.

As a corollary to these studies , we determined that the ‘control’ group of 50 current smokers (which were not diagnosed or currently suspected of having lung cancer) displayed an average HAAH serum level of essentially zero. However, two of these samples stood out as having a significantly elevated serum HAAH (12.0 and 18.7 ng/mL, respectively), which accounted for the decreased specificity of the test (90%) amongst smokers. As no follow-up of these individuals has yet been performed, it is unknown, yet interesting to speculate as to the true disease status of these individuals with regard to lung cancer.

### Example 3

In this study, we first compared HAAH serum levels in NSCLC patients versus healthy control individuals. Subsequently, a follow-on study of this biomarker was made in order to determine whether levels of the HAAH protein in serum can be correlated to clinical response to treatment.

Using the double monoclonal antibody sandwich ELISA described above, HAAH levels were measured in the sera of 46 patients with non-small cell lung cancer (NSCLC) and 15 healthy controls. A subsequent study, which measured serum HAAH levels both before and after treatment in 22 of the patients was conducted.

### Results

Serum HAAH was detectable and variable in 92% of NSCLC patients (n=43). Serum HAAH was found to be virtually undetectable in healthy donors. When patients were compared to healthy controls, the pretreatment median level of serum HAAH was significantly higher in NSCLC patients (P=0.00059). It was also noted that HAAH levels did not correlate with gender or age.

At the end of the treatment phase, an overall decrease of HAAH levels in the test population was observed. The results could be classified according to individual clinical response, by observing that pre-treatment HAAH levels were higher in patients who had progressive disease despite treatment (non-responders, n=19) as compared to responders (n=9) or stable disease patients (n=14). At the end of the treatment phase, a decrease of serum HAAH levels was particularly noted in the responder patients.

### Conclusion

The results suggest that the determination of pre-treatment serum HAAH levels is helpful to physicians in allowing the stratification of patients according to their likely clinical responsiveness to treatment and follow-up prognosis.

The heart of the invention and a number of embodiments of it have been fully described above, and highlighted in the claims below. It is considered understood by the reader that various modifications to the claimed invention may be made without departing from the spirit and scope of the invention described herein and as encompassed by the appended claims.

We claim:

1. A method for identifying whether a subject has lung cancer, comprising: contacting a serological sample of the subject with an anti-AAH antibody in vitro, under conditions that will allow specific immunological binding to occur with AAH in the sample, and detecting the level of anti-AAH/AAH immunocomplexes thus formed, whereby a detectable level of AAH in the serological sample is indicative of the presence of lung cancer in the subject.

2. The method of claim 1, wherein said serological sample is serum, and said level of AAH in the sample is at least about 3.2 ng/ml or higher.

3. The method of claim 1, wherein said serological sample is serum, and said level of AAH in the sample is at least about 3.7 ng/ml or higher.

4. The method of claim 1, wherein the level of AAH is determined by an enzyme-linked immunosorbant assay (ELISA) format.

5. The method of claim 1, which allows for the clinical monitoring of a therapeutic treatment for a patient with lung cancer, and further comprises: a) obtaining a serological sample at a first time point from a patient prior to or undergoing said treatment; b) repeating step a) at determined time points during the course of treatment, whereby the therapeutic treatment is temporally monitored by detecting any changes in expression of HAAH, and
wherein a decreased level of the AAH over time is associated with success of the therapeutic treatment, and an increased AAH level over time is indicative failure of the treatment.

6. The method of claim 1 or claim 5, wherein the level of AAH is determined by an immunoassay that employs two homologous antibodies, one of which is biotinylated.

7. The method of claim 1 or claim 5, wherein said subject is human, and the AAH is human AAH.

8. The method of claim 7, wherein said anti-AAH antibody is a mouse monoclonal antibody, which specifically binds with human AAH.

9. A method to determine whether a potential therapeutic agent is an effective drug for the treatment of lung cancer, comprising: a) establishing a human lung cancer tumor in a laboratory animal model; b) obtaining a serological sample at a determined first time point from the animal model at a time prior to instituting treatment with the therapeutic agent; c) detecting the level of AAH in said sample by assaying for human AAH protein with an immunological test format using one or more anti-HAAH antibodies; and d) repeating steps b) and c) at determined time points during the course of treatment with said therapeutic agent, whereby the effectiveness of said agent temporally monitored by detecting any changes in expression of HAAH, and
wherein a decreased level of the HAAH over time is associated with positive success of the therapeutic agent, and an increased HAAH level over time is indicative of failure of the therapeutic agent.

10. The method of claim 9, wherein the level of AAH is determined by an enzyme-linked immunosorbant assay (ELISA) format.

11. The method of claim 9, wherein the level of HAAH is determined by an immunoassay that employs two homologous antibodies, one of which is biotinylated.

12. The method of claim 11, wherein at said anti-AAH antibody is a mouse monoclonal antibody, which specifically binds with human AAH.

13. A test kit or device for the immunological determination of a level of AAH protein in a serological sample from a patient known or unknown to have lung cancer, comprising one or more anti-AAH antibodies, which will specifically bind to AAH, and instructions for performing the test and interpreting the results.

14. The device of claim 13, which is in the form of a dipstick or biochip, which device is composed of a solid support onto which the one or more specifically binding anti-HAAH antibodies is (are) bound, wherein at least one of said antibodies is labeled with a detectable label, and further situated within said dipstick or biochip is one or more compounds with which to detect the detectable label after a determined time of contact with a serological sample from a patient suspected of or known to have lung cancer, to thereby observe or measure the formation of immunological complexes of the anti-HAAH antibody and HAAH in the serological sample which, if detectable, is indicative of a positive result.

15. The device of claim 14, which consists of one anti-HAAH antibody, which is detectably labeled, and which acts as both capture and detection antibody.
